SEQUENCE LISTING (3 sequences)

SEQ ID NO: 1 - RNA, 20 nt, synthetic construct
  modified_base 18: mod_base = OTHER; note = Thymine
  modified_base 11: mod_base = OTHER; note = Thymine
  source 1..20: mol_type = other RNA; note = IgH sgRNA sequence
caaaacgcagtcccgcatcg

SEQ ID NO: 2 - RNA, 20 nt, synthetic construct
  modified_base 2: mod_base = OTHER; note = Thymine
  modified_base 9: mod_base = OTHER; note = Thymine
  modified_base 12: mod_base = OTHER; note = Thymine
  modified_base 14: mod_base = OTHER; note = Thymine
  modified_base 18: mod_base = OTHER; note = Thymine
  source 1..20: mol_type = other RNA; note = AAVS1 sgRNA sequence
gtcaccaatcctgtccctag

SEQ ID NO: 3 - RNA, 20 nt, synthetic construct
  modified_base 9..10: mod_base = OTHER; note = Thymine
  source 1..20: mol_type = other RNA; note = Universal sgRNA sequence
gggaggcgttcgggccacag

(19) United States
(12) Patent Application Publication
(10) Pub. No.: US 2025/0049850 A1
(43) Pub. Date: Feb. 13, 2025

(54) NON-VIRAL B CELL ENGINEERING METHODS

(71) Applicant: REGENTS OF THE UNIVERSITY OF MINNESOTA, Minneapolis, MN (US)

(72) Inventors: Branden S. Moriarity, Minneapolis, MN (US); Kanut Laoharawee, Minneapolis, MN (US); Beau R. Webber, Minneapolis, MN (US); Evan Kleinboehl, Minneapolis, MN (US)

(21) Appl. No.: 18/720,158

(22) PCT Filed: Dec. 15, 2022
(86) PCT No.: PCT/US22/81637

§ 371 Date: Jun. 14, 2024

Related U.S. Application Data
(60) Provisional application No. 63/289,858, filed on Dec. 15, 2021.

(51) Int. Cl. A61K 35/17; C07K 14/705; C12N 5/0781; C12N 9/22; C12N 9/24; C12N 15/86; C12N 15/90

(52) U.S. Cl. CPC A61K 35/17; C07K 14/70503; C12N 5/0635; C12N 9/22; C12N 9/2402; C12N 15/86; C12N 15/907; C07K 2319/60; C12N 2310/20; C12N 2510/00; C12N 2750/14143; C12N 2830/15; C12N 2840/44; C12Y 302/01076

(57) ABSTRACT

The present disclosure relates, in general, to non-viral methods for generating engineered B cell, and use of such B cells as cell-based therapeutics to treat disease.

### CROSS-REFERENCE TO RELATED APPLICATIONS

The present application claims the priority benefit of U.S. Provisional Patent Application No. 63/289,858, filed Dec. 15, 2021, hereby incorporated by reference.

### REFERENCE TO THE SEQUENCE LISTING

This application includes a sequence listing submitted electronically, in a file entitled: 57453_Seqlisting.xml created on Nov. 28, 2022 and having a size of 8,292 bytes, which is incorporated by reference herein.

### FIELD OF THE DISCLOSURE

The present disclosure relates, in general, to methods of making engineered B cells using non-viral based methods for modifying a B cell genome and use thereof as a therapeutic to treat disease.

### BACKGROUND

B lymphocytes, or B cells, are adaptive immune cells derived from lymphoid-lineage progenitors of hematopoietic stem cells in the bone marrow. B cells express at least CD20 and CD19 on the surface of the cells. Upon activation via B-cell receptors (BCRs), they differentiate further into long-lived plasma cells (CD138), and produce large amounts of different classes of antibodies, i.e., immunoglobulin alpha (IgA), delta (IgD), epsilon (IgE), gamma (IgG), and mu (IgM) as a part of immune response against foreign antigens such as bacterial or viral infections, cancers, parasites. The ability of differentiating into long-lived cells and produce large quantity of protein (antibodies) makes B cells attractive candidate to use as a cell-based gene therapy.

The precise genetic modification of primary human B cells has multiple applications in the fields of immunotherapy for cancers, infections, autoimmune diseases, and enzymopathies. Genetic modification of patient B cells is an attractive avenue for therapy due to the persistence of treatment and the low risks of rejection by the patient. Recombinant adeno-associated viral vectors (rAAV) a small DNA virus, with packaging capacity of 4.7 kilobase (Kb), have been used extensively as a vehicle for a DNA template delivery for immune cells, including B cells. A major drawback associated with rAAV is the small cargo capacity (4.7 kb), impeding researchers to use a larger therapeutic DNA template. In addition, several drawbacks associated with rAAV modification exist, for example, viral vector production can be costly and have a long turnaround time and several studies have reported immune response to rAAV, leading to rapid clearance of the rAAV-transduced cells (1,2).

### SUMMARY

The present disclosure provides non-viral based methods for editing the genome of a B cell that provide advantages over previous techniques. The genome edited or engineered B cell can overexpress a protein of interest or overexpress an endogenous gene and can be used as a therapeutic to treat various conditions and disorders.

The disclosure herein provides a method for genome engineering a B cell or a population of B cells to overexpress a gene of interest comprising introducing into the B cell or B cell population a plasmid, nanoplasmid or mini-circle comprising an expression cassette comprising a homology arm (HA), a polynucleotide encoding a gene of interest, a splice acceptor site, a promoter and a targeting site for a nuclease dependent cleavage system targeting molecule, wherein the expression cassette is inserted at a locus targeted by the targeting molecule.

Also provided is a method for genome engineering a B cell or a population of B cells to overexpress an endogenous gene comprising introducing into the B cell or B cell population a plasmid, nanoplasmid or mini-circle comprising an expression cassette comprising a homology arm(s) (HA), a splice acceptor site, a promoter and a targeting site for a nuclease dependent cleavage system targeting molecule, wherein the expression cassette is inserted upstream of a target gene to be overexpressed.

In various embodiments, the homology arms are between 35 and 1000 nucleotides. In various embodiments, the homology arms are 35, 40, 45, 50, 55, 60, 65, 70, 75, 80, 90, 100, 125, 150, 175, 200, 250, 300, 350, 400, 450, 500, 550, 600, 650, 700, 750, 800, 850, 900, 950 or 1000 nucleotides.

In various embodiments, the nuclease dependent cleavage system comprises a CRISPR/Cas system, a Cas-CLOVER system, a zinc-finger nuclease (ZFN) system, a transcription activator like effector nuclease (TALEN) system, or a meganuclease system. In various embodiments, the CRISPR/Cas system comprises Cas9, Cas12a, Cas13a or Cas13b.

In various embodiments, the nuclease dependent cleavage system is a CRISPR/Cas system and the targeting molecule is a guide RNA.

In various embodiments, the method further comprises transfecting the B cell or population of B cells with a Cas protein or polynucleotide encoding a Cas protein and guide RNA molecules that direct integration of the expression cassette to a target locus in the B cell genome.

In various embodiments, the method further comprises introducing into the B cell or B cell population a polynucleotide encoding a biomarker molecule useful to enrich for the B cell or a population of B cells. In various embodiments, the biomarker molecule comprises a fragment of CD34 and a fragment of CD20. In various embodiments, the biomarker polynucleotide is on the same expression cassette as the homology arm(s), splice acceptor site, targeting site for a nuclease dependent cleavage system targeting molecule and promoter.

In various embodiments, the gene of interest integrates into the B cell genome via homology directed repair (HDR), homology-mediated end joining (HMEJ) or a combination of HDR/HMEJ.

In various embodiments, the introduction of the plasmid, nanoplasmid or mini-circle is by transfection or electroporation. In various embodiments, efficiency of introduction is greater than 15%. In various embodiments, the efficiency of introduction is greater than 17%, 20%, 25%, 30%, 35%, 40%, 45%, 50%, 55% or more. In various embodiments, the B cell population has a viability of greater than 60%, 70%, 80%, 90% or more after 3 days. In various embodiments, the B cell population has a viability of greater than 60% after 3 days.

In various embodiments, the B cell or population of B cells is a primary B cell, a plasma cell, a mature B cell, a B cell line, a naïve B cell, memory B cells, or plasmablast.

In various embodiments, the plasmid or nanoplasmid comprises the promoter next to or near the gene of interest. In various embodiments, the promoter is an exogenous B cell promoter. In various embodiments, the promoter is an MND promoter, a CMV promoter, a CAG promoter, a PGK promoter, a EF1A promoter, a FEEK promoter, or a B cell-specific promoter.

In various embodiments, the gene of interest is a therapeutic gene or encodes a therapeutic protein. In various embodiments, the therapeutic gene encodes an enzyme, a cancer antigen, a cytokine, a chemokine, a B cell receptor, a cell surface receptor, or an antibody.

In various embodiments, the gene of interest is a donor polynucleotide that corrects a mutated genotype in a subject.

In various embodiments, the plasmid or nanoplasmid further comprises a polynucleotide encoding a B cell receptor or fragment thereof.

Also contemplated by the disclosure is a method of making a gene edited B cell or population of B cells, comprising: i) contacting a B cell or population of B cells with a plasmid, nanoplasmid or mini-circle comprising an expression cassette comprising a homology arm (HA), a splice acceptor site, a promoter and a targeting site for a nuclease dependent cleavage system targeting molecule, and optionally comprising a polynucleotide encoding a gene of interest; ii) culturing the B cell or population of B cells of i) in a media that promotes expansion of B cells; iii) isolating the B cell or population of B cells of ii) based on identification of a marker expressed only on a B cell or population of B cells carrying the plasmid or nanoplasmid; iv) culturing the isolated cells of iii) in a culture medium to expand the isolated cells expressing the gene of interest.

In various embodiments, the method further comprises a step of stimulating, proliferating or activating the B cell or population of B cells prior to the contacting step. In various embodiments, the step of stimulating, proliferating or activating the B cell or population of B cells comprises contacting the cell(s) with one or more of CpG, LPS, CD40L, Anti-IgM, IL-10, IL-15, IL-4, IL-2, IL-6, IFNα, APRIL or BAFF.

In various embodiments, the method produces gene edited B cells with an efficiency of greater than 15%. In various embodiments, the efficiency of introduction is greater than 17%, 20%, 25%, 30%, 35%, 40%, 45%, 50%, 55% or more. In various embodiments, the B cell population has a viability of greater than 60%, 70%, 80%, 90% or more after 3 days. In various embodiments, the B cell population has a viability of greater than 60% after 3 days.

In various embodiments, the culturing comprises use of culture media supplemented with CpG, or culturing media without CpG.

In various embodiments, the expression cassette is between 500 nucleotides and 10 kilobases (kb). In various embodiments, the expression cassette is between 1000 nucleotides and 8 kB, between 1500 nucleotides and 7 kb, between 2 kb and 6.5 kb, larger than 4.7 kb, or 1 kb, 1.5 kb, 2 kb, 2.5 kb, 3 kb, 3.5 kb, 4 kb, 4.5 kb, 5 kb, 5.5 kb, 6 kb, 6.5 kb, 7 kb, 7.5 kb, 8 kb, 8.5 kb, 9 kb, 9.5 kb or 10 kb, including all numeric value within the ranges and endpoints of said ranges.

Also provided is a gene edited B cell or population of B cells made by the methods described herein.

Also provided is a gene edited B cell comprising: i) a heterologous polynucleotide sequence encoding a gene of interest integrated in the B cell genome at a target location mediated by a nuclease dependent cleavage system, wherein the heterologous polynucleotide sequence is also flanked by portions of a homology arm and expressed via an endogenous promoter; and ii) a heterologous biomarker molecule. In various embodiments, the B cell is a primary B cell, a plasma cell, a B cell line, naïve B cells, memory B cells, or a plasma blast.

In various embodiments, the gene of interest is a therapeutic gene or encodes a therapeutic protein. In various embodiments, the therapeutic gene encodes an enzyme, a cancer antigen, a cytokine, a chemokine, a B cell receptor, a cell surface receptor, or an antibody. In various embodiments, the gene of interest is a donor polynucleotide that corrects a mutated genotype in a subject.

Further contemplated is a method of treating a disease or condition in a subject in need thereof comprising administering to the subject a gene edited B cell or population of B cells as described herein. In various embodiments, the disease is an enzymopathy, a cancer, a precancerous condition, an infection, or a genetic disorder.

In various embodiments, the disease is an enzymopathy. In various embodiments, the enzymopathy is selected from the group consisting of aspartylglucosaminuria, cholesterol ester storage disease, Wolman disease, metachromatic leukodystrophy, Danon disease, Fabry disease, Farber lipogranulomatosis, Farber disease, fucosidosis, galactosialidosis types I/II, Gaucher disease types I/II/III, globoid cell leukodystrophy, Krabbe disease, glycogen storage disease II, Pompe disease, GM1-gangliosidosis types I/II/III, GM2-gangliosidosis type I, Tay Sachs disease, GM2-gangliosidosis type II, Sandhoff disease, GM2-gangliosidosis, α-mannosidosis types I/II, β-mannosidosis, mucolipidosis type I, sialidosis types I/II, mucolipidosis types II/III, I-cell disease, mucolipidosis type IIIC, pseudo-Hurler polydystrophy, mucopolysaccharidosis type I, mucopolysaccharidosis type II, Hunter syndrome, mucopolysaccharidosis type IIIA, Sanfilippo syndrome, mucopolysaccharidosis type IIIB, mucopolysaccharidosis type IIIC, mucopolysaccharidosis type IIID, mucopolysaccharidosis type IVA, mucopolysaccharidosis type IVB Morquio syndrome, mucopolysaccharidosis type VI, mucopolysaccharidosis type VII, Sly syndrome, mucopolysaccharidosis type IX, multiple sulfatase deficiency, neuronal ceroid lipofuscinosis, CLN1 Batten disease, CLN2 Batten disease, Niemann-Pick disease types A/B, Niemann-Pick disease, Niemann-Pick disease type C1, Niemann-Pick disease type C2, pycnodysostosis, Schindler disease types I/II, and sialic acid storage disease, hemophilia A, hemophilia B, Christmas disease, and Factor VII deficiency.

In various embodiments, the enzymopathy is mucopolysaccharidosis type I (MPS I) and the gene of interest is iduronidase.

In various embodiments, the cancer is a solid tumor or a blood cancer. In various embodiments, the cancer is selected from the group consisting of acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head and neck cancer, Hodgkin's lymphoma, Kaposi's sarcoma, kidney cancer, liver cancer, lung cancer (small and non-small cell), malignant peritoneal effusion, malignant pleural effusion, melanoma, mesothelioma, multiple myeloma, neuroblastoma, glioma, non-Hodgkin's lymphoma, osteosarcoma, ovarian cancer, ovarian (germ cell) cancer, pancreatic cancer, penile cancer, prostate cancer, retinoblastoma, skin cancer, soft tissue sarcoma, squamous cell carcinomas, stomach cancer, testicular cancer, thyroid cancer, trophoblastic neoplasms, uterine cancer, vaginal cancer, cancer of the vulva, and Wilms's tumor.

In various embodiments, the genetic disorder is selected from the group consisting of muscular dystrophy, cystic fibrosis, Sickle cell anemia, β-thalassemia, a lysosomal storage disorder, Adenosine Deaminase Deficiency, Severe Combined Immunodeficiency (SCID), Retinitis Pigmentosa, macular degeneration, and Wiskott-Aldrich Syndrome.

In various embodiments, the B cell is first isolated from the subject to be treated and then genetically modified according to a method described herein. In various embodiments, the isolated, genetically modified B cell is replaced into the subject from which it was derived.

Further aspects and advantages will be apparent to those of ordinary skill in the art from a review of the following detailed description, taken in conjunction with the drawings. While the compositions, articles, and methods are susceptible of embodiments in various forms, the description hereafter includes specific embodiments with the understanding that the disclosure is illustrative, and is not intended to limit the invention to the specific embodiments described herein. For the compositions, articles, and methods described herein, optional features, including but not limited to components, compositional ranges thereof, substituents, conditions, and steps, are contemplated to be selected from the various aspects, embodiments, and examples provided herein.

### BRIEF DESCRIPTION OF THE DRAWINGS

FIG. 1A-1B. A viral-based approach (CRISPR/Cas9 together with recombinant AAV (rAAV) carried EGFP donor template) mediated efficient EGFP knock-in in B cells. (FIG. 1A) a construct comprised of gene trap constructs (a splice acceptor (Splice Acc.), follows by poly adenylation, MND promoter and EGFP coding sequence flanked on both sides with 1000 base-pair (bps) homology arms for insertion at IgH locus. This construct was packaged into rAAV6 viral vector to use as a donor template for EGFP insertion at the IgH locus of the B cells. (FIG. 1B) CRISPR/Cas9 with the rAAV mediated efficient transgene insertion in B cells (up to 64%). No or low percent GFP-positive cells was observed in either control or B cells transduced with vector only.

FIG. 2. Comparing a therapeutic construct and the original EGFP construct on the rAAV backbone for viral-based engineering. (Top) the original EGFP construct. (Bottom) the therapeutic construct was made by simply replacing EGFP coding sequence with IDUA-2A-RQR8. IDUA is a therapeutic gene. RQR8, a domain of CD34 or CD20 that doesn't exist in B cells, is used for engineered-cell identity, and can be used to enrich engineered cells from bulk population. Due to the large therapeutic construct, the homology arms were reduced to 400 bps to meet a 4.7-kilobase packaging capacity of rAAV vector.

FIG. 3. The therapeutic constructs with reduced homology arms resulted in drastically decreased viral-based engineering efficiency. The reduced homology arms (400 bps) of the therapeutic IDUA construct (FIG. 2) hindered efficient large transgene insertion (% RQR8-positive) in B cells when compared to of the small transgene (EGFP) construct that can utilize the optimal 1000-bp homology arms.

FIGS. 4A-4B. Plasmid can be efficiently introduced and well tolerated in B cells. pMax GFP plasmid was electroporated (transfected) in B cells. (FIG. 4A) Up to 80% GFP-positive B cells was observed post pMax GFP plasmid transfection. (FIG. 4B) B cells showed slight reduced % viability post transfection when compared to electroporated cells without plasmid (control).

FIGS. 5A-5C. Comparison of three constructs to compare mechanisms; Homology directed repair (HDR), a hybrid HMEJ/HDR, and HMEJ (Homology mediate end-joining), for precise insertion of the transgene on the B-cell genome. (FIG. 5A) The HDR construct will act as a donor template in a circular fashion. (FIG. 5B) HMEJ/HDR construct is resembling the HDR construct with additional synthetic CRISPR/Cas9 target sequence (namely, universal gRNA target sequence, in gray bar at termini). Upon transfection of CRISPR/Cas9 with universal gRNA, the construct will be cleaved on both sides of the homology arms, resulting linearized HMEJ/HDR template for transgene insertion. (FIG. 5C) HMEJ construct is similar to the HMEJ/HDR construct, but with only 48-bp homology arms.

FIG. 6. Efficient gene transfer was observed across mechanisms regardless of the size of homology arms. Plasmids in FIG. 5 were individually transduced in B cells to compare engineering efficiencies. All mechanisms for insertion showed similar engineering efficiencies with slightly higher in HDR/HMEJ and slightly lower in HMEJ methods. High engineering efficiency of the small homology arms (48-bp) of the HMEJ construct suggests linearized donor template mitigate the need of large homology arms.

FIGS. 7A-7B. Overall B-cell health was affected by non-viral approach, but B cells rebound quickly. The non-viral engineering approach affected overall B-cell health (viability (FIG. 7A), and cell recovery (FIG. 7B)). HDR method was less abrasive to B cells when compared to HMEJ or the hybrid methods. Overall, B cells in all mechanisms started to rebound at day 5 post engineering.

FIG. 8A-8B. Therapeutic constructs to compare engineering efficiency of viral-based and non-viral-based approaches. Two identical constructs were made on (FIG. 8A) rAAV for viral-based approach and on (FIG. 8B) a Nanoplasmid™ for non-viral approach. These therapeutic constructs utilize 250-bp homology arms, the plasmid constructs have additional synthetic CRISPR/Cas9 target sequence as previously described in FIG. 5.

FIG. 9A-9B. Non-viral approach showed higher engineering efficiencies, leading to higher expression of the therapeutic gene. B cells were engineered using either viral- or non-viral approach to compare engineering efficiencies. (FIG. 9A) The non-viral approach enhances large transgene insertion in B cells (up to 18% RQR8-positive cells) when compared to the viral approach (up to 6% RQR8-positive cells). (FIG. 9B) the higher engineering efficiency from the non-viral approach leads to higher expression of the therapeutic gene (IDUA).

FIG. 10. Cell cycle analysis of B cell cultured in immunocult. HDR and HMEJ are active during Late G1/S/G2 phase of the cell cycle. Highest G1/S/G2 phase was observed from 48-, 60-, and 72-hr stimulation time.

FIG. 11A-11B. B cell health of 48-, 60- and 70-hr stimulation time point at 48 hours post engineering (n=3). B cells were stimulated for 48-, 60- or 72-hr before engineering. At 48-hr post engineering, the cells were sampled to count to determine cell recovery and viability as indications for B-cell health. (FIG. 11A) 80% viability was observed across the samples regardless of stimulation time. (FIG. 11B) Cell recovery was exceeded the number of input (1E⁺⁶; dash line).

FIG. 12. RQR8+ B cells of different stimulation time at day 5 post engineering (n=3). To determine engineering efficiency, RQR8 was co-expressed with the IDUA therapeutic protein. RQR8 is a domain of CD34 and CD20 that is not express in B cells, which will be tagged on the cell surface of the engineered B cells that can be easily detected by flow cytometer. 18% RQR8-positive B cells were observed in the HDR/HMEJ sample of 48-hr stimulation time, while 60- and 72-hr showed insignificant decrease of engineering rates. HDR sample showed only 5% RQR8-positive B cells all across the stimulation times. No RQR8-positive cells were observed in Vector only and control.

FIG. 13A-D. Non-viral, HMEJ engineering of human B-cells with 6.2 kb cargo. (FIG. 13A) Diagram of large, 6.2 kb multicistronic construct for targeted integration at AAVS1 locus; the construct comprises MesoCAR-2A-RQR8-BGH-MND-CD19-DHFR-GFP-BGH-2. (FIG. 13B) Integration efficiencies when using HMEJ for a targeted knock-in (KI). (FIG. 13C) B cell viability post engineering. (FIG. 13D) B cell quantities post engineering.

### DETAILED DESCRIPTION

This method(s) described herein provide insertion of single or multiple gene coding sequences of proteins, e.g., cancer antigens, specific B cell receptors (BCRs), antibodies, or enzymes in primary human B cells, memory B cells, B-cell lines, plasma blasts, and plasma cells. This protocol may also eliminate drawbacks associated with rAAV vector as previously described. This protocol will allow high engineering efficiencies with little or no negative effect on B cell health post engineering. The engineered cells can be used as a cell-based therapy for protein deficiencies, enzymopathies, immunotherapy for infections, autoimmune diseases, and cancers. Provided herein is a protocol for a non-viral genome engineering approach for B cells using plasmids, Nanoplasmid™ or mini-circle as a donor template to enhance nuclease-dependent genome editing systems-mediated site-specific insertion of a large transgene in B cells.

### Definitions

Unless otherwise defined, all technical and scientific terms used herein have the same meaning as commonly understood by one of ordinary skill in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

Each publication, patent application, patent, and other references cited herein is incorporated by reference in its entirety to the extent that it is not inconsistent with the present disclosure.

It is noted here that as used in this specification and the appended claims, the singular forms “a,” “an,” and “the” include plural reference unless the context clearly dictates otherwise.

“Amplification” refers to any means by which a polynucleotide sequence is copied and thus expanded into a larger number of polynucleotide molecules, e.g., by reverse transcription, polymerase chain reaction, and ligase chain reaction.

“cDNA” refers to a DNA that is complementary or identical to an mRNA, in either single stranded or double stranded form.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is identical to the nucleotide sequence of the polynucleotide binding partner of the second polynucleotide. Thus, the polynucleotide whose sequence 5′-TATAC-3′ is complementary to a polynucleotide whose sequence is 5′-GTATA-3′. A nucleotide sequence is “substantially complementary” to a reference nucleotide sequence if the sequence complementary to the subject nucleotide sequence is substantially identical to the reference nucleotide sequence.

“Conservative substitution” refers to the substitution in a polypeptide of an amino acid with a functionally similar amino acid. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

The term “fragment” when used in reference to polypeptides refers to polypeptides that are shorter than the full-length polypeptide by virtue of truncation at either the N-terminus or C-terminus of the protein or both, and/or by deletion of an internal portion or region of the protein. Fragments of a polypeptide can be generated by methods known in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Expression control sequence” refers to a nucleotide sequence in a polynucleotide that regulates the expression (transcription and/or translation) of a nucleotide sequence operatively linked thereto. “Operatively linked” refers to a functional relationship between two parts in which the activity of one part (e.g., the ability to regulate transcription) results in an action on the other part (e.g., transcription of the sequence). Expression control sequences can include, for example and without limitation, sequences of promoters (e.g., inducible or constitutive), enhancers, transcription terminators, a start codon (i.e., ATG), splicing signals for introns, and stop codons.

The term “promoter” as used herein refers to a region of DNA that functions to control the transcription of one or more DNA sequences, and that is structurally identified by the presence of a binding site for DNA-dependent RNA-polymerase and of other DNA sequences, which interact to regulate promoter function. A functional expression promoting fragment of a promoter is a shortened or truncated promoter sequence retaining the activity as a promoter. Promoter activity may be measured in any of the assays known in the art e.g. in a reporter assay using Luciferase as reporter gene (Wood, 1991; Seliger and McElroy, 1960; de Wet et al. (1985), or commercially available.

The term “vector” refers to any carrier of exogenous DNA or RNA that is useful for transferring exogenous DNA to a host cell for replication and/or appropriate expression of the exogenous DNA by the host cell. “Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in vitro expression system. Expression vectors include all those known in the art, such as cosmids, plasmids (e.g., naked or contained in liposomes), nanoplasmids or mini-circles that incorporate the recombinant polynucleotide.

“Nanoplasmid™” refers to a 500 base-pair circular plasmid lacking traditional bacterial genes or antibiotic resistant genes, which in turn reduces cellular toxicity and inflammation of the plasmid-transfected cells, when compared to traditional plasmid. Mini-circles similarly lack bacterial genes.

“Expression cassette” or “cassette” refers to a component of vector or plasmid DNA that controls expression of a gene or protein, and may be interchangeable and easily inserted or removed from a vector. Expression cassettes often comprise a promoter sequence, an open reading frame, and a 3′ untranslated region that contains a polyadenylation site.

An “enhancer region” refers to a region of DNA that functions to increase the transcription of one or more genes. More specifically, the term “enhancer”, as used herein, is a DNA regulatory element that enhances, augments, improves, or ameliorates expression of a gene irrespective of its location and orientation. It is contemplated that an enhancer may enhance expression of more than one promoter.

“Polynucleotide” refers to a polymer composed of nucleotide units. Polynucleotides include naturally occurring nucleic acids, such as deoxyribonucleic acid (“DNA”), including cDNA, and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. The term “protein” typically refers to large polypeptides. The term “peptide” typically refers to short polypeptides. Conventional notation is used herein to portray polypeptide sequences: the left-hand end of a polypeptide sequence is the amino-terminus; the right-hand end of a polypeptide sequence is the carboxyl-terminus.

“Recombinant polynucleotide” refers to a polynucleotide having sequences that are not naturally joined together. An amplified or assembled recombinant polynucleotide may be included in a suitable vector, and the vector can be used to transform a suitable host cell. A host cell that comprises the recombinant polynucleotide is referred to as a “recombinant host cell.” The gene is then expressed in the recombinant host cell to produce, e.g., a “recombinant polypeptide.” A recombinant polynucleotide may serve a non-coding function (e.g., promoter, origin of replication, ribosome-binding site, etc.) as well. Recombinant protein refers to a protein encoded by a recombinant polynucleotide.

“Substantially pure” or “isolated” means an object species is the predominant species present (i.e., on a molar basis, more abundant than any other individual macromolecular species in the composition), and a substantially purified fraction is a composition wherein the object species comprises at least about 50% (on a molar basis) of all macromolecular species present. Generally, a substantially pure composition means that about 80% to 90% or more of the macromolecular species present in the composition is the purified species of interest. The object species is purified to essential homogeneity (contaminant species cannot be detected in the composition by conventional detection methods) if the composition consists essentially of a single macromolecular species. Solvent species, small molecules (<500 Daltons), stabilizers (e.g., BSA), and elemental ion species are not considered macromolecular species for purposes of this definition. In some embodiments, the lysosomal sulfatase enzymes of the invention are substantially pure or isolated. In some embodiments, the lysosomal sulfatase enzymes of the invention are substantially pure or isolated with respect to the macromolecular starting materials used in their synthesis. In some embodiments, the pharmaceutical composition of the invention comprises a substantially purified or isolated therapeutic lysosomal sulfatase enzyme admixed with one or more pharmaceutically acceptable carriers, diluents or excipients.

The term “specifically binds” is “antigen specific”, is “specific for”, “selective binding agent”, “specific binding agent”, “antigen target” or is “immunoreactive” with an antigen refers to a B cell receptor or polypeptide that binds a target antigen with greater affinity than other antigens of related proteins.

The term “endogenous” refers to a protein, polynucleotide, or other molecule that is naturally found in or expressed by a subject, e.g., a cell, organ, or tissue. The term “exogenous” refers to a protein, polynucleotide, or other molecule that is not naturally found in a subject, e.g., a cell, organ, or tissue.

The term “genetically engineered” as used herein refers to a polynucleotide or polypeptide sequence that has been modified from its naturally-occurring sequence, e.g., by insertion, deletion or polynucleotide or amino acid substitution/modification, using recombinant DNA expression techniques to produce a polypeptide or polynucleotide sequence that differs from the previously unmodified sequence.

The term “nuclease dependent cleavage system” as used herein refers to gene editing techniques that employ DNA or RNA dependent nucleases to cleave target DNA or RNA, respectively, and molecules or guides that direct the nuclease to the target DNA/RNA to be cleaved. Examples of nuclease dependent cleavage systems include CRISPR/Cas systems, Cas-CLOVER systems, zinc-finger nuclease (ZFN) systems, transcription activator like effector nuclease (TALEN) systems, or meganuclease systems.

“Homozygous” for the donor polynucleotide as used herein refers to the result of the genetic modification in which both alleles of the modified gene express the donor polynucleotide. “Heterozygous” for the donor polynucleotide as used herein refers to the result of the genetic modification in which only one of the alleles of the gene express the donor polynucleotide.

### B Cells

B cells can become long lived and inherently have the ability to generate large quantities of protein (i.e. antibody), and could provide an useful platform for gene therapy including, for example, for treatment of enzymopathies, cancer or other disorders. B cells are also readily available in peripheral blood (making up 1-7% of all leucocytes), and methods to expand the cells are readily available. Moreover, data suggest that cells cross the blood brain barrier more readily than proteins, potentially making cellular therapies for enzymopathies with brain involvement more desirable than enzyme replacement therapy. Yet the delivery of therapeutic genes to B cells using genome-engineering approaches or the use of any targeted nuclease in primary human B cells has not previously been reported. Methods for engineering B cells using viral based methods are disclosed in co-owned application U.S. Ser. No. 16/332,555.

The present disclosure provides methods of engineering B cells that do not rely on viral vectors for introducing genetic material.

In various embodiments, the B cell can be a CD19⁺ cell. In various embodiments, the B cell can be a primary B cell. As used herein, a “primary B cell” is a non-immortalized B cell. A primary B cell can be a B cell that is freshly isolated, or the primary B cell is isolated from peripheral blood mononuclear cells (PBMCs). In various embodiments, the B cell is derived from an induced pluripotent stem cell (iPSC). In various embodiments, the B cell is derived from a population of CD34⁺ cells.

In various embodiments, a “primary B cell” is a B cell that has undergone up to 5 replications or divisions after being isolated, up to 10 replications or divisions after being isolated, up to 15 replications or divisions after being isolated, up to 20 replications or divisions after being isolated, up to 25 replications or divisions after being isolated, up to 30 replications or divisions after being isolated, up to 35 replications or divisions after being isolated, or up to 40 replications or divisions after being isolated.

In various embodiments, a “primary B cell” is a B cell that has undergone up to 5 replications or divisions after being derived, up to 10 replications or divisions after being derived, up to 15 replications or divisions after being derived, up to 20 replications or divisions after being derived, up to 25 replications or divisions after being derived, up to 30 replications or divisions after being derived, up to 35 replications or divisions after being derived, or up to 40 replications or divisions after being derived.

In various embodiments, the primary B cell is a non-clonal cell. In various embodiments, primary B cell is a proliferating cell. In various embodiment the B cell is preferably cultured in the presence of CD40L.

In various embodiments, the B cell is a naïve B cell. In various embodiments, a “naïve B cell” is CD19⁺, IgD⁺, IgM⁺, CD27⁻, CD21⁺, and/or CXCR5⁺. In various embodiments, the B cell is a memory B cell. In various embodiments, a “memory B cell” is CD19⁺, IgD⁻, CD27⁺ or CD27⁻, CD21⁺, and/or CXCR5⁺. In various embodiments, the B cell is an activated memory B cell. In various embodiments, an “activated memory B cell” is CD19⁺, IgD⁻, CD27⁺, CD21⁻, and/or CXCR5⁺. In various embodiments, the B cell is a natural effector B cell. In various embodiments, a “natural effector B cell” is CD19⁺, IgD⁺, IgM⁺, and/or CD27⁺. In various embodiments, the B cell is a plasmablast. In various embodiments, a “plasmablast” is CD19⁺, CXCR5⁻, CD38⁺, CD27⁻, and/or CD20⁻.

In various embodiments, the B cell is a B cell that has undergone class-switch recombination. In some embodiments, the B cell is a B cell that has not undergone class-switch recombination.

In various embodiments, the B cell is a mammalian cell. In certain embodiments, the B cell is a human cell. In other embodiments, the B cell is a mouse cell.

A B cell is “genome edited” or “engineered” if the B cell includes a modification to its genome compared to a non-genome edited B cell. In various embodiments, a non-genome edited B cell is a wild-type B cell. In various embodiments, a non-genome edited B cell is a freshly isolated B cell.

In various embodiments, the genome edited B cell includes a modification of a noncoding region of the genome and/or a coding region of the genome (e.g., a gene). In various embodiments, the noncoding region of the genome can include a sequence for a small, regulatory noncoding RNA, including, for example, a microRNA (miRNA). In various embodiments, the noncoding region of the genome is preferably involved in regulating the function, activation, and/or survival of the B cell.

In various embodiments, an engineered B cell includes a modification that alters expression or activity of the engineered B cell relative to a non-engineered B cell. For example, in various embodiments, the engineered B cell may include an expression cassette, as described herein.

### Genome Engineering

CRISPR/Cas and other nuclease-based gene editing systems open a new avenue to altering a gene of interest by creating double stranded breaks (DSB), leading to formation of small insertions or deletions created by semi-random repair via the Non-Homologous End Joining (NHEJ) pathway. Alternatively, precise genome modifications can be achieved by the introduction of CRISPR/Cas9 to induce a DSB along with a DNA template for Homology directed repair (HDR). A DNA template can be designed to encode a transgene of interest such as for B cell receptor (BCR) or encode a therapeutic protein/enzyme that can be used for cancer immunotherapy or protein/enzyme deficiency, respectively.

In various embodiments, the method includes a technique to introduce a protein or nucleic acid into the B cell or population of B cells. Any suitable method of introducing a protein or nucleic acid may be used. In some embodiments, the method preferably includes electroporation of a B cell or population of B cells to introduce genetic material including, for example, DNA, RNA, and/or mRNA. As used herein, electroporation may include nucleofection. Because plasmid DNA can be toxic to B cells, in some embodiments, mRNA or protein based approaches of genome editing are preferred. In some embodiments, a technique to introduce a protein or nucleic acid can include introducing a protein or nucleic acid via electroporation; microinjection; exosomes; liposomes; biolistics; jet injection; hydrodynamic injection; ultrasound; magnetic field-mediated gene transfer; electric pulse-mediated gene transfer; use of nanoparticles including, for example, lipid-based nanoparticles; incubation with an endosomolytic agent; use of cell-penetrating peptides; etc. In some embodiments, the method preferably includes electroporation of a B cell using a NEON transfection system, Lonza transfection system, or MaxCyte transfection system.

In various embodiments, the method includes editing a gene. Editing a gene can include introducing one or more copies of the gene, altering the gene, deleting the gene, upregulating expression of the gene, downregulating expression of the gene, mutating the gene, methylating the gene, demethylating the gene, acetylating the gene, and/or deacetylating the gene. Mutating the gene can include introducing activing mutations, introducing inactivating and/or inhibitory mutations, and/or introducing point mutations.

In some embodiments, the method preferably includes inducing double stranded breaks in the genome of the B cell. Double stranded breaks may be introduced using a nuclease dependent cleavage systems including, for example, a transcription activator-like effector nucleases (TALEN), a zinc finger nuclease (ZFN), a CRISPR-associated nuclease, etc.

If a CRISPR/Cas system is used, it includes use of a guide RNA (gRNA) or DNA (gDNA) targeting molecule. The gRNA target or gDNA target can include any suitable target. In various embodiments, the target includes a portion of the B cell genome including, for example, a gene or a portion of a gene.

The disclosure herein provides a method for genome engineering a B cell or a population of B cells to overexpress a gene of interest comprising introducing into the B cell or B cell population a plasmid or nanoplasmid comprising an expression cassette comprising a homology arm (HA), a polynucleotide encoding a gene of interest, a splice acceptor site, a promoter and a targeting site for a nuclease dependent cleavage system targeting molecule, wherein the expression cassette is inserted at a locus targeted by the targeting molecule.

Also provided is a method for genome engineering a B cell or a population of B cells to overexpress an endogenous gene comprising introducing into the B cell or B cell population a plasmid, nanoplasmid or mini-circle comprising an expression cassette comprising a homology arm(s) (HA), a splice acceptor site, a promoter and a targeting site for a nuclease dependent cleavage system targeting molecule, wherein the expression cassette is inserted upstream of a target gene to be overexpressed.

In some embodiments, where transfection may be used to deliver a CRISPR/Cas9 system, the gRNA may preferably include a chemically modified gRNA. In some embodiments, the chemical modification to the gRNA preferably decreases a cell's ability to degrade the RNA. In some embodiments, a chemically modified gRNA includes one or more of the following modifications: 2′-fluoro (2′-F), 2′-O-methyl (2′-O-Me), S-constrained ethyl (cEt), 2′-O-methyl (M), 2′-O-methyl-3′-phosphorothioate (MS), and/or 2′-O-methyl-3′-thiophosphonoacetate (MSP). In some embodiments, the chemically modified gRNA can include a gRNA and/or a chemical modification described in Hendel et al, Nature Biotechnology, 2015, 33(9):985-989 or Rahdar et al., PNAS, 2015, 112(51):E7110-7.

It is contemplated that the present method provides efficient transfer of the gene of interest and provides improved viability of the B cells after genome modification. For example, the efficiency of transfer of the gene of interest, or overexpression of an endogenous polynucleotide is 10%, 15%, 20%, 25%, 30%, 35%, 40%, 45%, 50%, 55%, 60%, 65%, 70%, 75% or more. In various embodiments, the B cell population has a viability of greater than 60%, 70%, 80%, 90% or more after 3 days. In various embodiments, the B cell population has a viability of greater than 60% after 3 days.

In some embodiments, the method includes selecting a gene edited B cell. In some embodiments, the selection is performed after editing a gene. A B cell can, in some embodiments, be selected using one or more of the following methods: flow sorting (including, for example, for cell surface marker expression); magnetic bead separation (including, for example, targeting a cell-surface marker); transient drug resistance gene expression (including, for example, antibiotic resistance).

In some embodiments, the method includes expanding an edited B cell. In some embodiments, the expansion can be performed after selecting the edited B cell. In some embodiments, a B cell can be expanded by co-incubation with an antigen recognized by the B cell receptor or a cell expressing an antigen recognized by the B cell receptor. In some embodiments, a B cell can be expanded (e.g., stimulating, proliferating or activating) by co-incubation with a cytokine or ligand including, for example, contacting the cell(s) with one or more of CpG, LPS, CD40L, Anti-IgM, IL-10, IL-15, IL-2, IL-6, IL-4, IFNα, APRIL or BAFF.

In some embodiments, the B cell can be stimulated for at least 18 hours, at least 1 day, at least 36 hours, at least 2 days, at least 3 days, at least 4 days, at least 5 days, at least 6 days, or at least 7 days. In some embodiments, the B cell can be simulated for up to 1 day, up to 2 days, up to 3 days, up to 4 days, up to 5 days, up to 6 days, up to 7 days, up to 8 days, up to 9 days, up to 10 days, up to 12 days, up to 14 days, up to 3 weeks, up to 4 weeks, or up to two months. In some embodiments, the B cell is preferably stimulated for 14 days.

### Nuclease Dependent Cleavage Systems

Zinc-finger nucleases (ZFNs) and Transcription activator-like effector nucleases (TALENs) are customizable DNA-binding proteins that comprise DNA-modifying enzymes. Both can be designed and targeted to specific sequences in a variety of organisms (Esvelt and Wang, Mol Syst Biol. (2013) 9: 641). ZFNs and TALENs are useful to introduce a broad range of genetic modifications by inducing DNA double-strand breaks that stimulate error-prone non-homologous end joining (NHEJ) or homology-directed repair (HDR) at specific genomic locations. These DNA-binding modules can be combined with numerous effector domains to affect genomic structure and function, including nucleases, transcriptional activators and repressors, recombinases, transposases, DNA and histone methyltransferases, and histone acetyltransferases. Thus, the ability to execute genetic alterations depends largely on the DNA-binding specificity and affinity of designed zinc finger and TALEN proteins (Gaj et al., Trends in Biotechnology, (2013) 31(7):397-405). The following U.S. granted patents, incorporated by reference, describe the use of ZFNs and TALENs in mammalian cells, U.S. Pat. Nos. 8,685,737 and 8,697,853.

CRISPR-Cas (clustered regularly interspaced short palindromic repeats-CRISPR associated) is an RNA-mediated adaptive immune system found in bacteria and archaea, which provides adaptive immunity against foreign nucleic acids (Wiedenheft et al., Nature (2012) 482:331-8; Jinek et al., Science (2012) 337:816-21). Recent studies have shown that the biological components of this system can be used to modify to the genome of mammalian cells. CRISPR-Cas systems are generally defined by a genomic locus called the CRISPR array, a series of 20-50 base-pair (bp) direct repeats separated by unique “spacers” of similar length and preceded by an AT-rich “leader” sequence (Wright et al., Cell (2016) 164:29-44).

Three types of CRISPR/Cas systems exist, type I, II and III. The Type II CRISPR-Cas systems require a single protein, e.g., Cas9, to catalyze DNA cleavage (Sapranauskas et al., Nucleic Acids Res. (2011) 39(21): 9275-9282). Cas9 serves as an RNA-guided DNA endonuclease. Cas9 generates blunt double-strand breaks (DSBs) at sites defined by a 20-nucleotide guide sequence contained within an associated CRISPR RNA (crRNA) transcript. Cas9 requires both the guide crRNA and a trans-activating crRNA (tracrRNA) that is partially complementary to the crRNA for site-specific DNA recognition and cleavage (Deltcheva et al., Nature (2011)4 71(7340):602-7; Jinek et al., Science (2012) 337:816-21).

The crRNA:tracrRNA complex can be synthesized as two separate molecules or as a single transcript (single-guide RNA or sgRNA) encompassing the features required for both Cas9 binding and DNA target site recognition. Using sgRNA, Cas from bacterial species, such as S. pyogenes, can be programmed to cleave double-stranded DNA at any site defined by the guide RNA sequence and including a protospacer-adjacent (PAM) motif (Sapranauskas et al., Nucleic Acids Res. (2011) 39(21): 9275-9282; Jinek et al., Science (2012) 337:816-21). The DSBs result in either non-homologous end-joining (NHEJ), which is error-prone and conducive to frameshift mutations that knock out gene alleles, or homology-directed repair (HDR), which can be exploited with the use of an exogenously introduced double-strand or single-strand DNA repair template to knock in or correct a mutation in the genome. Therefore, in the presence of a homologous repair donor, the CRISPR/Cas9 system may be used to generate precise and defined modifications and insertions at a targeted locus through the HDR process. In the absence of a homologous repair donor, single DSBs generated by CRISPR/Cas9 are repaired through the error-prone NHEJ, which results in insertion or deletion (indel) mutations.

Other publications describing the CRISPR systems and Cas9, include the following Cong et al. Science (2013) 339:819-23; Jinek et al., eLife 2013; 2:e00471. (2013) 2:e00471; Lei et al. Cell (2013) 152: 1173-1183; Gilbert et al. Cell (2013) 154:442-51; Lei et al. eLife (2014) 3:e04766; Perez-Pinela et al. Nat Methods (2013) 10: 973-976; Maider et al. Nature Methods (2013) 10, 977-979 which are incorporated by reference. The following U.S. and international patents and patent applications describe the methods of use of CRISPR, U.S. Pat. Nos. 8,697,359; 8,771,945; 8,795,965; 8,865,406; 8,871,445; 8,889,356; 8,895,308; 8,906,616; 8,932,814; 8,945,839; 8,993,233; 8,999,641; 2014/0068797; and WO 2014/197568, each of which is incorporated by reference in their entirety.

The CRISPR related protein, Cas9, can be from any number of species including, but not limited to, Streptococcus pyogenes, Staphylococcus aureus, Listeria innocua, and Streptococcus thermophilus.

Additional Cas proteins known in the art are contemplated for use in the methods, including Cas12a (Cpf1) and Cas13a/Cas13b (56). See also Yan et al., Cell Biology and Toxicology 35:489-492 (2019).

Cas-CLOVER™ systems are recently designed gene editing systems that utilize the Clo51 nuclease instead of the CRISPR protein. Cas-CLOVER™ comprises a nuclease-inactivated Cas9 protein fused to the Clo51 endonuclease. Cas-CLOVER uses two guide RNAs as well as a nuclease activity that requires dimerization of subunits associated with each guide RNA to provide target specificity.

In one embodiment, the methods use a CRISPR-Cas system and one or more guide RNAs, repair templates and HDR to insert nucleotide bases into the genome of a B cell locus.

### Nucleic Acid Molecules

Nucleic acids of the disclosure can be cloned into a vector, such as a plasmid, nanoplasmid or mini-circle, into which another genetic sequence or element (either DNA or RNA) may be inserted so as to bring about the replication of the attached sequence or element. In various embodiments, the expression vector contains a constitutively active promoter segment (such as but not limited to CMV, SV40, Elongation Factor or LTR sequences) or an inducible promoter sequence such as the steroid inducible pIND vector (Invitrogen), where the expression of the nucleic acid can be regulated. Expression vectors of the invention may further comprise regulatory sequences. The vector can be introduced into a cell or embryo by transfection, for example.

A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. For instance, in some embodiments, signal peptide sequences may be appended/fused to the amino terminus of any of the donor polynucleotide, CRISPR-Cas or other nuclease-dependent cleavage system described herein.

A vector may also comprises a nucleic acid comprising a promoter, a coding sequence of a transgene of interest, optionally a poly A sequence, and homology arms. In various embodiments, the nucleic acid comprises homology arm(s) (HA), a splice acceptor site, a promoter and a targeting site for a nuclease dependent cleavage system targeting molecule.

In various embodiments, the homology arms are between 35 and 1000 nucleotides. In various embodiments, the homology arms are 35, 40, 45, 50, 55, 60, 65, 70, 75, 80, 90, 100, 125, 150, 175, 200, 250, 300, 350, 400, 450, 500, 550, 600, 650, 700, 750, 800, 850, 900, 950 or 1000 nucleotides.

In various embodiments, the plasmid or nanoplasmid comprises a nucleic acid further comprising a promoter next to or near the gene of interest/gene to be overexpressed. In various embodiments, the promoter is an endogenous B cell promoter. In various embodiments, the promoter is an MND promoter, a CMV promoter, a CAG promoter, a PGK promoter, a EF1A promoter, a FEEK promoter, or a B cell-specific promoter.

### Cell Culture Methods

Mammalian B cells containing the recombinant protein-encoding DNA or RNA are cultured under conditions appropriate for growth of the cells and expression of the DNA or RNA. Those cells which express the recombinant protein can be identified, using known methods and methods described herein, and the recombinant protein can be isolated and purified, using known methods and methods also described herein, either with or without amplification of recombinant protein production. Identification can be carried out, for example, through screening genetically modified mammalian cells that display a phenotype indicative of the presence of DNA or RNA encoding the recombinant protein, such as PCR screening, screening by Southern blot analysis, or screening for the expression of the recombinant protein. Selection of cells which contain incorporated recombinant protein-encoding DNA may be accomplished by including a selectable marker in the DNA construct, with subsequent culturing of transfected or infected cells containing a selectable marker gene, under conditions appropriate for survival of only those cells that express the selectable marker gene. Further amplification of the introduced DNA construct can be effected by culturing genetically modified B cells under appropriate conditions (e.g., culturing genetically modified B cells containing an amplifiable marker gene in the presence of a concentration of a drug at which only cells containing multiple copies of the amplifiable marker gene can survive).

Genetically modified B cells expressing the recombinant protein can be identified, as described herein, by detection of the expression product or cell surface markers.

Protein purification methods are known in the art and utilized herein for recovery of recombinant proteins from cell culture media. For example, methods of protein and antibody purification are known in the art and can be employed with production of the antibodies of the present disclosure. In some embodiments, methods for protein and antibody purification include filtration, affinity column chromatography, cation exchange chromatography, anion exchange chromatography, and concentration. The filtration step may comprise ultrafiltration, and optionally ultrafiltration and diafiltration. Filtration is preferably performed at least about 5-50 times, more preferably 10 to 30 times, and most preferably 14 to 27 times. Affinity column chromatography, may be performed using, for example, PROSEP® Affinity Chromatography (Millipore, Billerica, Mass.). In various embodiments, the affinity chromatography step comprises PROSEP®-vA column chromatography. Eluate may be washed in a solvent detergent. Cation exchange chromatography may include, for example, SP-Sepharose Cation Exchange Chromatography. Anion exchange chromatography may include, for example but not limited to, Q-Sepharose Fast Flow Anion Exchange. The anion exchange step is preferably non-binding, thereby allowing removal of contaminants including DNA and BSA. The antibody product is preferably nanofiltered, for example, using a Pall DV 20 Nanofilter. The antibody product may be concentrated, for example, using ultrafiltration and diafiltration. The method may further comprise a step of size exclusion chromatography to remove aggregates.

### Methods of Use

The engineered B cell of the present disclosure are useful to as a cell-based therapy for protein deficiencies, enzymopathies, immunotherapy for infections, autoimmune diseases, and cancers.

In various embodiments, the engineered B cell(s) comprises an expression cassette comprising a polynucleotide to be overexpressed by the B cell. In various embodiments, the engineered B cell comprises an exogenous polynucleotide integrated within the genome.

In various embodiments, the engineered B cell comprises an expression cassette comprising a polynucleotide to be overexpressed by the B cell or an exogenous polynucleotide, wherein the polynucleotide encodes an antibody, a cytokine, a chemokine, a B cell receptor, or a cell surface receptor. In various embodiments, the polynucleotide encodes an antigen that is a cancer antigen, a tumor specific antigen, a neo antigen, an enzyme, an autoimmune antigen, a microbial antigen, a viral antigen, or a bacterial antigen.

In various embodiments, the enzyme is an enzyme deficient in an enzymopathy. In various embodiments, the enzyme is L-Iduronidase, Iduronate-2-sulfatase, Heparan-N-sulfatase α-N-Acetylglucosaminidase AcetylCoA: N-acetyltransferase, N-Acetylglucosamine 6-sulfatase, Galactose 6-sulfatase, βGalactosidase, N-Acetylgalactosamine 4-sulfatase, β-Glucuronidase, hyaluronoglucosaminidase, Aspartylglucosaminidase, Acid lipase, Cystine transporter, Lamp, α-Galactosidase A. ceramidase, α-L-Fucosidase, Protective protein, β-glucosidase, Galactocerebrosidase, α-Glucosidase, β-Galactosidase, β-Hexosaminidase A, α-D-Mannosidase, β-D-Mannosidase, Arylsulfatase A, Neuraminidase, Saposin B, Phosphotransferase, Phosphotransferase γ-subunit, Palmitoyl protein thioesterase, Tripeptidyl peptidase I, Acid sphingomyelinase, Cathepsin K, α-Galactosidase B, sialic acid transporter, Factor VII, or Factor VIII.

In various embodiments, the enzymopathy is selected from the group consisting of aspartylglucosaminuria, cholesterol ester storage disease, Wolman disease, metachromatic leukodystrophy, Danon disease, Fabry disease, Farber lipogranulomatosis, Farber disease, fucosidosis, galactosialidosis types I/II, Gaucher disease types I/II/III, globoid cell leukodystrophy, Krabbe disease, glycogen storage disease II, Pompe disease, GM1-gangliosidosis types I/II/III, GM2-gangliosidosis type I, Tay Sachs disease, GM2-gangliosidosis type II, Sandhoff disease, GM2-gangliosidosis, α-mannosidosis types I/II, β-mannosidosis, mucolipidosis type I, sialidosis types I/II, mucolipidosis types II/III, I-cell disease, mucolipidosis type IIIC, pseudo-Hurler polydystrophy, mucopolysaccharidosis type I, mucopolysaccharidosis type II, Hunter syndrome, mucopolysaccharidosis type IIIA, Sanfilippo syndrome, mucopolysaccharidosis type IIIB, mucopolysaccharidosis type IIIC, mucopolysaccharidosis type IIID, mucopolysaccharidosis type IVA, mucopolysaccharidosis type IVB Morquio syndrome, mucopolysaccharidosis type VI, mucopolysaccharidosis type VII, Sly syndrome, mucopolysaccharidosis type IX, multiple sulfatase deficiency, neuronal ceroid lipofuscinosis, CLN1 Batten disease, CLN2 Batten disease, Niemann-Pick disease types A/B, Niemann-Pick disease, Niemann-Pick disease type C1, Niemann-Pick disease type C2, pycnodysostosis, Schindler disease types I/II, and sialic acid storage disease, hemophilia A, hemophilia B, Christmas disease, and Factor VII deficiency.

In various embodiments, the enzymopathy is mucopolysaccharidosis type I (MPS I) and the gene of interest to be overexpressed is iduronidase.

In various embodiments, the cancer antigen in mesothelin, BCMA, CD19, CD20, CD22, CD70, CD123, CEA, CDH3, CLDN6, CLL1, CS1, DCAF4L2, FLT3, GABRP, MageB2, MART-1, MSLN, MUC1 (e.g., MUC1-C), MUC12, MUC13, MUC16, mutFGFR3, PRSS21, PSMA, RNF43, STEAP1, STEAP2, TM4SF5, PD-1, CTLA4, EGFR, VEGF, OX40, or FcRL5.

In various embodiments, the cancer is a solid tumor or a blood cancer. In various embodiments, the cancer is selected from the group consisting of leukemias, brain tumors (including meningiomas, glioblastoma multiforme, anaplastic astrocytomas, cerebellar astrocytomas, other high-grade or low-grade astrocytomas, brain stem gliomas, oligodendrogliomas, mixed gliomas, other gliomas, cerebral neuroblastomas, carniopharyngiomas, diencephalic gliomas, germinomas, medulloblastomas, ependymomas. choroid plexus tumors, pineal parenchymal tumors, gangliogliomas, neuroepithelial tumors, neuronal or mixed neuronal glial tumors), lung tumors (including small cell carcinomas, epidermoid carcinomas, adenocarcinomas, large cell carcinomas, carcinoid tumors, bronchial gland tumors, mesotheliomas, sarcomas or mixed tumors), prostate cancers (including adenocarcinomas, squamous cell carcinoma, transitional cell carcinoma, carcinoma of the prostatic utricle, or carcinosarcomas), breast cancers (including adenocarcinomas or carcinoid tumors), or gastric, intestinal, or colon cancers (including adenocarcinomas, invasive ductal carcinoma, infiltrating or invasive lobular carcinoma, medullary carcinoma, ductal carcinoma in situ, lobular carcinoma in situ, colloid carcinoma or Paget's disease of the nipple), skin cancer (including melanoma, squamous cell carcinoma, tumor progression of human skin keratinocytes, basal cell carcinoma, hemangiopericytoma and Karposi's sarcoma), lymphoma (including Hogkin's disease and non-Hodgkin's lymphoma), and sarcomas (including osteosarcoma, chondrosarcoma and fibrosarcoma).

In various embodiments, the autoimmune antigen is associated with an autoimmune disease. In various embodiments, the autoimmune disease is selected form the group consisting of achalasia, Addison's disease, adult still's disease, agammaglobulinemia, alopecia areata, amyloidosis, ankylosing spondylitis, anti-gbm/anti-tbm nephritis, antiphospholipid syndrome autoimmune angioedema autoimmune dysautonomia autoimmune encephalitis autoimmune hepatitis, autoimmune inner ear disease (AIED), autoimmune myocarditis autoimmune oophoritis, autoimmune orchitis, autoimmune pancreatitis, autoimmune retinopathy autoimmune urticarial, axonal & neuronal neuropathy (AMAN), Baló disease, Behcet's disease, benign mucosal pemphigoid (Mucous membrane pemphigoid), bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatosis (EGPA), cicatricial pemphigoid, Cogan's syndrome, cold agglutinin disease, complex regional pain syndrome (formerly known as reflex sympathetic dystrophy), congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (acne inversa), IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, lichen planus, lichen sclerosus, ligneous conjunctivitis, linear IgA disease (LAD), lupus, Lyme disease chronic, Meniere's disease, microscopic polyangiitis (MPA), mixed connective tissue disease (MCTD), Mucha-Habermann disease, multifocal motor neuropathy (MMN) or MMNCB, multiple sclerosis, myasthenia gravis, myelin oligodendrocyte glycoprotein antibody disorder, myositis, narcolepsy, neonatal lupus, neuromyelitis optica/device disease, neutropenia, ocular cicatricial pemphigoid, optic neuritis, palindromic rheumatism (PR), PANDAS (Pediatric autoimmune neuropsychiatric disorders associated with Streptococcus infections), paraneoplastic cerebellar degeneration (PCD), paroxysmal nocturnal hemoglobinuria (PNH), pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatic, polymyositis, postmyocardial infarction syndrome, postpericardiotomy syndrome, primary biliary cholangitis, primary sclerosing cholangitis, progesterone dermatitis, progressive hemifacial atrophy (PHA), Parry romberg syndrome, psoriasis, psoriatic arthritis, pure red cell aplasia (PRCA), pyoderma gangrenosum, Raynaud's phenomenon, reactive arthritis, relapsing polychondritis, restless legs syndrome (RLS), retroperitoneal fibrosis, rheumatic fever, rheumatoid arthritis, sarcoidosis, Schmidt syndrome or autoimmune polyendocrine syndrome type II, scleritis, scleroderma, Sjögren's Disease, stiff person syndrome (SPS), Susac's syndrome, sympathetic ophthalmia (SO), Takayasu's arteritis, temporal arteritis/giant cell arteritis, thrombocytopenic purpura (TTP), thrombotic thrombocytopenic purpura (Ttp), thyroid eye disease (Ted), Tolosa-Hunt syndrome (THS), transverse myelitis, type 1 diabetes, ulcerative colitis (UC), undifferentiated connective tissue disease (UCTD), uveitis, vasculitis, vitiligo, Vogt-Koyanagi-Harada disease, and warm autoimmune hemolytic anemia.

In some embodiments, the expression cassette includes a nucleic acid encoding a BCR. In some embodiments, the BCR is specific to an antigen that can be administered to a subject via immunization. In some embodiments, the BCR preferably includes a transmembrane region and/or a membrane bound-antibody. As used herein, a BCR may include either a membrane-anchored BCR or a soluble Ig or both.

In various embodiments, the expression cassette includes a nucleic acid that encodes a heavy chain and/or a light chain of an antibody. In some embodiments, the transcription of the polynucleotide encoding a heavy chain and/or light chain can be driven by an endogenous promote or an exogenous promoter.

In various embodiments, the expression cassette includes a nucleic acid that encodes a heavy chain and/or a light chain of a B cell receptor. In some embodiments, the transcription of the polynucleotide encoding a heavy chain and/or light chain can be driven by an endogenous promoter or an exogenous promoter. In various embodiments, the polynucleotide encoding the heavy chain and/or light chain encodes a single variable segment, a single diversity segment, a single joining segment, and a single C-region. In some embodiments, the polynucleotide encoding the heavy chain preferably encodes a transmembrane region including, for example, an M1 and/or an M2 domain.

In various embodiments, a engineered B cell includes an expression cassette comprising a polynucleotide encoding a cytokine. The cytokine includes, but is not limited to, IL-10, IL-4, IL-7, IL-2, IL-15, IL-6, IL-21, B cell activating factor (BAFF), a proliferation-inducting ligand (APRIL), IFN-α/β or IFN-γ, or combinations thereof.

In various embodiments, the method further comprises inactivating a gene encoding the target antigen of interest in the non-human animal. In various embodiments, the gene encoding the target antigen is inactivated using a nuclease-dependent cleavage system.

### Kits

The polynucleotides, plasmid system or vectors described herein may be provided in a kit. The kits may include, in addition to the polynucleotide, plasmid system or vector, any reagent which may be employed in the use of the system. In one embodiment, the kit includes reagents necessary for transformation of the vectors into mammalian cells. The kit may include growth media or reagents required for making growth media, for example, DMEM for growth of mammalian cells. Components supplied in the kit may be provided in appropriate vials or containers (e.g., plastic or glass vials). The kit can include appropriate label directions for storage, and appropriate instructions for usage.

### EXAMPLES

### Example 1—Materials and Methods

Culture media: ExCellerate™ recovery media: ExCellerate™ B cell media Xeno free (R&D Systems), 5% CTS Immune cell SR, 55 μM 2-mercaptoethanol. ExCellerate™ complete media: ExCellerate™ recovery media supplemented with 1 μg/mL CpG ODN 2006 (Invivogen), 100 ng/mL MEGA CD40 ligand, 50 ng/mL IL-10, and 10 ng/mL IL-15. Recovery media: ImmunoCult™ XF B cell base medium; ImmunoCult™ complete media comprised ImmunoCult™-XF B cell base medium supplement with 2× ImmunoCult™-ACF Human B Cell Expansion Supplement.

Cell culture: Stimulation protocol and engineering protocol are the same for both CpG-based or non-CpG-based media. All steps are performed with aseptic technique. Stimulation of B cells for engineering: Complete B cell media is pre-equilibrated at least 15 minutes before use. 2 mL of a complete B cell media is aliquoted into a 15 ml conical tube, incubated in 37° C. water bath for 10 minutes. Thaw cryopreserved B cells and immediately drip 1 mL of pre-warmed media from the 15 mL conical tube into the cryopreserve tube containing B cells. The whole volume of cell suspension is transferred from cryopreserve tube back to the 15 mL conical tube with pre-warmed media. Bring up the volume to 15 mL with 1×PBS and centrifuge at 400×g for 5 minutes. Remove supernatant without disturbing the pellet and then resuspend cells with a complete media to a concentration of 1×10⁶ cell/mL. Transfer the cell suspension into a T25 flask and place in the tissue culture incubator at 37° C., 5% CO₂ with humidity for 48 hours.

B cell engineering: Prior to engineering cells are counted 48 hours post simulation. Count cells and transfer 1×10⁶ cells per electroporation reaction into a 15 mL conical tube and centrifuge at 200×g for 10 minutes. Remove supernatant, resuspend the cell pellet with 14 mL 1×PBS and centrifuge at 200×g for 10 minutes, remove supernatant completely. Transfer protector RNase inhibitor from a stock solution into Nucleofection reagent at a final volume of 1:50 per electroporation reaction, mix well. Resuspend cell pellet with 20.46 uL per 1×10⁶ B cells. Transfer 20.46 uL of cell suspension to the PCR tubes containing 3 uL of substrate, do not mix. Transfer 23.46 uL of electroporation reaction from the PCR tube to a cuvette of a 16-cuvette strip, do not mix. Cap, tap gently to ensure the reaction goes down to the bottom of the cuvette. Electroporate using EO-117 (human B cell protocol) on Lonza 4D system. Rest the electroporated cells in the cuvette at room temperature for 15 minutes. Transfer 80 uL the pre-equilibrate recovery media into the electroporated cells and immediately transfer the cells to a well of 48-well plate containing 500 uL recovery media. If viral vectors are used, immediately transfer rAAV viral vector to the viral approach conditions at 250,000 multiples of infection (MOI). Place the plate back into the tissue culture incubator and incubate for 30 minutes. After 30 minutes, transfer 500 uL of 2× complete media into the well containing cells. The final concentration of the cells at this step is 1×10⁶ cell/mL. Place the plate back to the tissue culture incubator for 5-12 days. Keep the cells at 1×10⁶ cell/mL for 48 hours. Count cells, determine % viability, sampling cells for flow cytometry analysis for EGFP-positive B cells or RQR8-positive B cells to determine engineering efficiency at day 2- and day 5-post engineering.

CRISPR/Cas9: Chemically modified sgRNAs 1. IgH sgRNA sequence: CAAAACGCAGTCCCGCATCG (SEQ ID NO: 1)—targets IGHM locus of B cells; 2. AAVS1 sgRNA sequence: GTCACCAATCCTGTCCCTAG (SEQ ID NO: 2)—targets AAVS1 locus of B cells; 3. Universal sgRNA sequence: GGGAGGCGTTCGGGCCACAG (SEQ ID NO: 3)—targets Universal target sequence on a nanoplasmid for HMEJ mechanism; 3. CleanCap™ S.p. Cas9 mRNA.

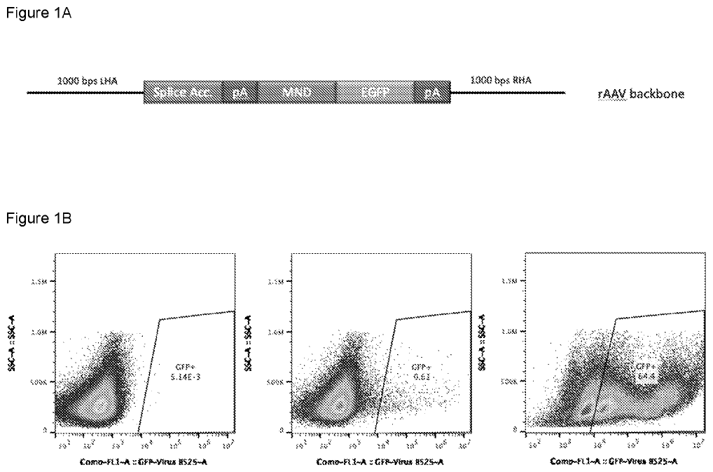

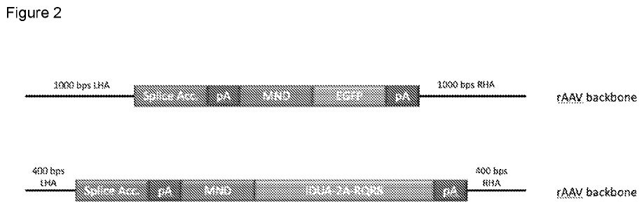

rAAV vector constructs: Targets IgH locus of the B cells; 1. rAAV MND-EGFP (FIG. 1); 2. rAAV MND-IDUA-RQR8 (FIG. 2, bottom); 3. rAAV SA-T2A-RQR8-MND-IDUA (FIG. 8).

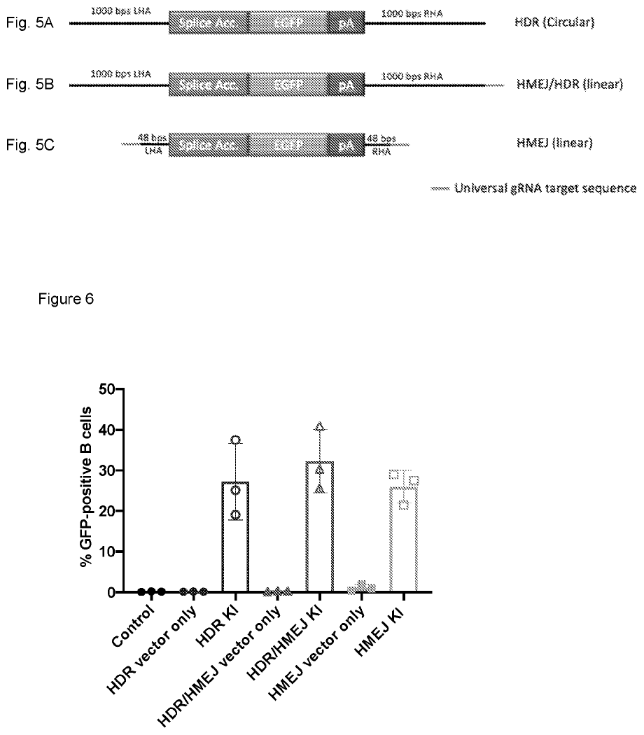

Nanoplasmid constructs: Targets AAVS1 locus; 1. HDR SA-GFP (FIG. 5A); 2. HMEJ/HDR SA-GFP (FIG. 5B); 3. HMEJ SA-GFP (FIG. 5C). Targets IgH locus; 1. HDR/HMEJ SA-T2A-RQR8-MND-IDUA (FIG. 8).

Electroporation: Lonza 4D electroporator system; AMAXA™ P3 Primary Cell 40-NUCLEOFECTOR™ X kit contains: 2×16-cuvette strips, P3 primary Cell Solution, Supplement 1, Protector RNase inhibitor (200 units/uL).

Flow cytometry: Cell are stained with CD19 (B cell marker), viability dye (eF780) and anti-CD34 antibody clone QBEND/10 conjugated with PE.

### Example 2-Generation and Characterization of Engineered B Cells

Previously, it was reported using rAAV vector as a vehicle to deliver a DNA template for CRISPR/Cas9 to mediate HDR for insertion of EGFP³. A typical DNA template for HDR often contains a promoter, a coding sequence of a transgene of interest, and 1000 base-pair homology arms. The DNA template is cloned into an AAV viral backbone that contain 5′- and 3′-inverted terminal repeats (ITRs). All together, the construct including ITRs must not exceed 4.7 Kb packaging capacity of the virus.

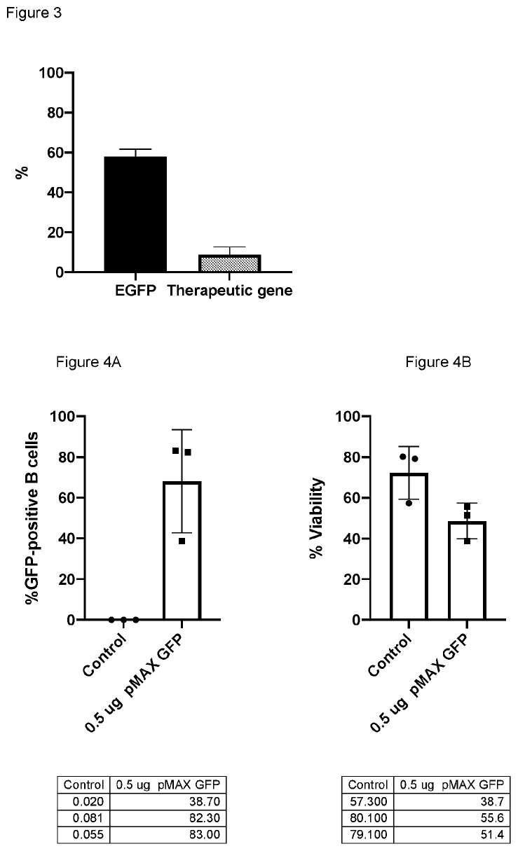

The present experiment utilized a small DNA template consisting of MND promoter and EGFP coding sequence (FIG. 1A), which allows for use of the optimal 1000 base pairs homology arms (FIG. 1), resulting in high engineering efficiencies (FIG. 1B)³. Once translated from a small EGFP construct to a larger construct containing DNA encoding therapeutic protein (IDUA) that co-express with RQR8 (FIGS. 2A and 2B), the homology arms were reduced to only 400 base pairs (FIG. 2B) to fit in the 4.7 kb packaging capacity of the rAAV. This small homology arms leads to drastic reduction of engineering efficiencies of the therapeutic construct when compared to the efficiency of the EGFP with 1000 base-pair homology arms (FIG. 3). This emphasizes a significant drawback of using a small rAAV vector to deliver a DNA template to a B cell. Therefore, the feasibility of using a non-viral approach such as a plasmid as a donor template for engineering in B cells was investigated.

First, it was tested whether B cells can tolerate plasmid transfection. B cells were stimulated with ExCellerate™ complete media for 48 hours prior to engineering. Upon 48-hour stimulation, B cells were transfected with pMax GFP plasmid via electroporation. Up to 80% transfection rates were observed indicated by % EGFP-positive B cells (FIG. 4A) with approximately 45% viability when compared to control (FIG. 4B). This indicates that plasmid can efficiently transfect B cells via electroporation; however, B cell health was somewhat affected by the process.

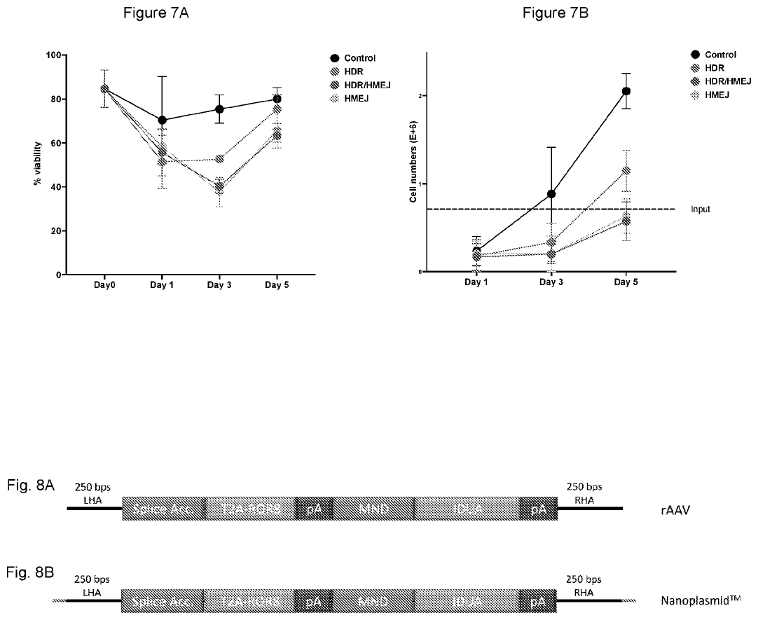

Next, several precise DNA repair mechanisms to insert EGFP on the B cell genome were investigated for optimal engineering efficiency. Three expression cassettes were constructed that differ only by the length of homology arms and with/without a universal gRNA target sequence. HDR constructs contain 1000 base-pair homology arms without the universal gRNA target sequence (FIG. 5A). HMEJ/HDR hybrid constructs contain 1000 base-pair homology arms and the universal gRNA target sequence (FIG. 5B). HMEJ constructs contain 48 base-pair homology arms and the universal gRNA target sequence (FIG. 5C). The universal gRNA target sequence is necessary for HMEJ mechanisms because this method utilizes linearized DNA template⁴. All of the constructs were cloned into Nanoplasmid™ backbone. Similar engineering efficiencies were observed amongst the tested DNA repair mechanism (FIG. 6), regardless of size of homology arms. Surprisingly, the HMEJ method with only 48 base-pair homology arms showed insignificant reductions when compared to its counterparts with 1000 base-pair homology arms. This indicates that HMEJ mitigates the need for large homology arms. Next it was tested whether B-cell health is affected by the process, as assessed by percent viability (FIG. 7A) and cell recovery (FIG. 7B) at 48-hours post engineering. It was found that all tested mechanisms affected B cell health. While HDR resulted in slightly better health of B cells, all B cells started to rebound after 5 days. Moving forward, HMEJ method was used for the subsequent experiments.

One attempt to improve engineering efficiency was to use RNase inhibitor. It was found that intracellular RNases can reduce RNA expression, resulting in lowering engineering efficiencies in other immune cells. This was tested by transfecting RNase inhibitor along with CRISPR/Cas9 and a plasmid to improve engineering efficiencies. Interestingly, enhanced engineering frequencies were not observed when using RNase inhibitor, but slightly better B cell health post engineering was observed. Moving forward, RNase inhibitor was used for subsequent experiments.

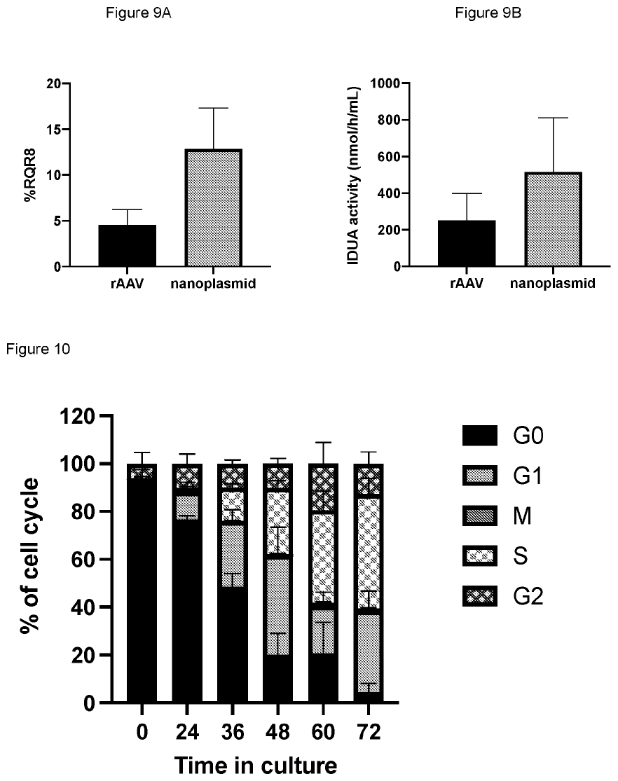

Next, 2 constructs were constructed containing DNA encoding a therapeutic gene that co-expressed with RQR8 and flanked both side of the constructs with 250 base-pair homology arms, the only differences between these constructs is the absence or present of universal gRNA target sequence (FIGS. 8A and 8B, respectively). The construct without universal gRNA target sequence was cloned into rAAV backbone (FIG. 8A; viral approach). The construct that contains universal gRNA target sequence was cloned into nanoplasmid backbone (FIG. 8B; non-viral approach). The viral approach was tested in parallel with the non-viral approach. Significantly higher engineering efficiency was observed when using the non-viral approach, while the viral approach resulted in much lower efficiency (FIG. 9A). The higher engineering efficiency leads to higher therapeutic gene expression (IDUA; FIG. 9B). This indicates that the non-viral approach using nanoplasmid enhanced efficiency of a large transgene insertion in B cells compared to a viral vector.

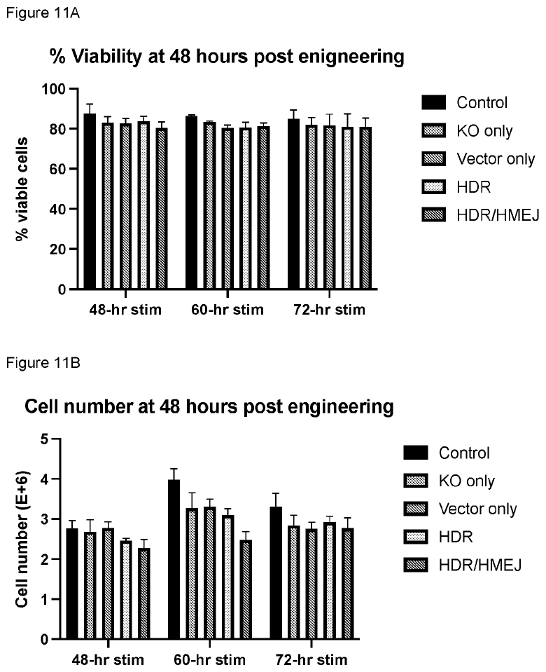

DNA sensing mechanisms have been reported as an innate immune response of cells⁵. TLR9 is one of the DNA sensing pathways, reported to be activated when CpG oligodeoxynucleotide is present in B cells⁶. Interestingly, one of the components in the ExCellerate™ media contains CpG ODN 2006, which activates TLR9 per manufacturer's claims. It was hypothesized that TLR9 may play a role in B cell health after plasmid transfection. Since CpG is indispensable for the ExCellerate™ complete media, a search for a media that doesn't activate TLR9 pathway was undertaken. A new media, “Immunocult”, a STEMCELL technology proprietary formula that does not activate TLR pathways was identified and tested for culture of engineered B cells. HDR has been reported to be active during S/G2 phase of the cell cycle, while HMEJ was speculated to be active during late G1/early S phase of the cell cycle⁴. A cell cycle analysis was conducted to determine the optimal time to engineer the cells. It was determined that the stimulation time point where G1/S/G2 phase is the most active will allow engineering of cells with the optimal engineering efficiencies. Little or no G1/S/G2 phases was observed during 0, 24, and 36 hours of stimulation, while drastic increase of these phases was observed at 48, 60, and 72 hours of stimulation (FIG. 10). Next, it was tested whether 48-, 60-, or 72-hr stimulation time points gives the optimal engineering efficiency by introducing CRISPR/Cas9 targeting IgH locus of B cells along with the therapeutic Nanoplasmid™ DNA template (FIG. 8B). Surprisingly, similar viability (80%) was observed between control and engineered cells (HDR, HDR/HMEJ; FIG. 11A). In addition, the cell recovery surpassed the 1×10⁶ input cell numbers (FIG. 11B). This suggests the cells can tolerate plasmid engineering process very well. Moving forward, B cells were engineered at these three time points.

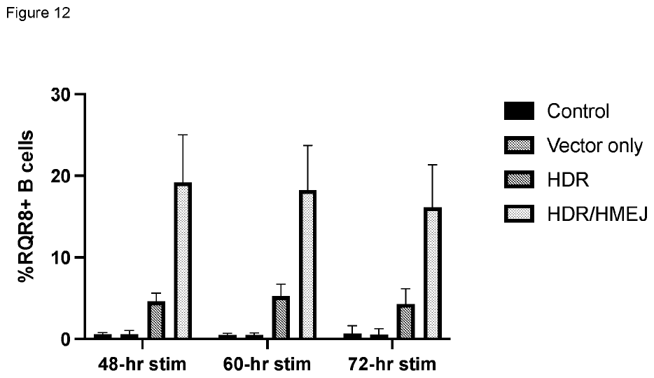

Next, the RQR8 was measured via flow cytometry to determine engineering efficiency. 5% engineering efficiency was observed in samples knocked-in using HDR mechanism, while 18% engineering efficiencies were observed in samples knocked-in using HDR/HMEJ mechanism across all stimulation time points (FIG. 12). This indicates that the non-TLRs stimulation media showed superior cell health and engineering efficiency for a non-viral approach to engineering B cells. In addition, the HDR/HMEJ mechanism enhanced transgene insertion when compared to HDR method.

In summary, the results demonstrate the non-viral engineering protocol using Nanoplasmid™ as a DNA template for CRISPR/Cas9 mediated insertion of a large transgene resulted in optimal engineering efficiencies. In addition, non-CpG media gave the optimal cell health and engineering efficiencies for a non-viral approach for B-cell engineering.

### Example 3—Non-Viral, HMEJ Engineering of Human B-Cells with Large Cargo

rAAV has a small cargo capacity of 4.7 kb, which limits the ability to use larger therapeutic DNA templates in the vectors. In order to determine the capability of non-viral based engineering to deliver constructs exceeding the 4.7 kb limitation of rAAV, engineered B cells were developed with larger polynucleotide cargo sizes.

CRISPR/Cas9 reagents were made by mixing 1 μL (1 μg/μL) of chemically modified sgRNA and 1.5 μL (1 μg/μL) of chemically-modified Streptococcus pyogenes Cas9 (S.p. Cas9) Nuclease mRNA for a total of 2.5 μL CRISPR/Cas9 reagent per 1×10⁶ B cells.

B cells were engineered as described above. For transfection, cells were suspected in P3 primary cell transfection reagent to a concentration of 1×106 cells/20 μL and transferred into a PCR tube. 2.5 μL CRISPR/Cas9 reagent was transferred into the 20 μL cell suspension and mixed gently and transferred to an electroporation cuvette (Lonza 4D) and transfected as above. Rest the electroporated cells in the cuvette at room temperature for 15 minutes. 80 uL the pre-equilibrate recovery media was transferred into the electroporated cells and placed in tissue culture incubator for 30 min. The cells were transferred to a well of 48-well plate containing 1 mL B cell expansion media for a final concentration of 5×10⁵ cells/ml. Place the plate in tissue culture incubator for 5-12 days at 37° C., 5% CO₂. B cell expansion medium was refreshed every 2 days by transferring the whole volume of the cells into a clean 15 mL conical tube, centrifuge at 400×g for 5 minutes, discard supernatant discarded without disturbing the pellet, and cells resuspended with 100 uL fresh B cell expansion medium and transferred to a tissue culture plate, maintaining a cell concentration at 5×10⁵ cells/mL. Cells were counted and viability recorded at day 1 post engineering. The engineered cells were expanded for at least 5 days before downstream analysis.

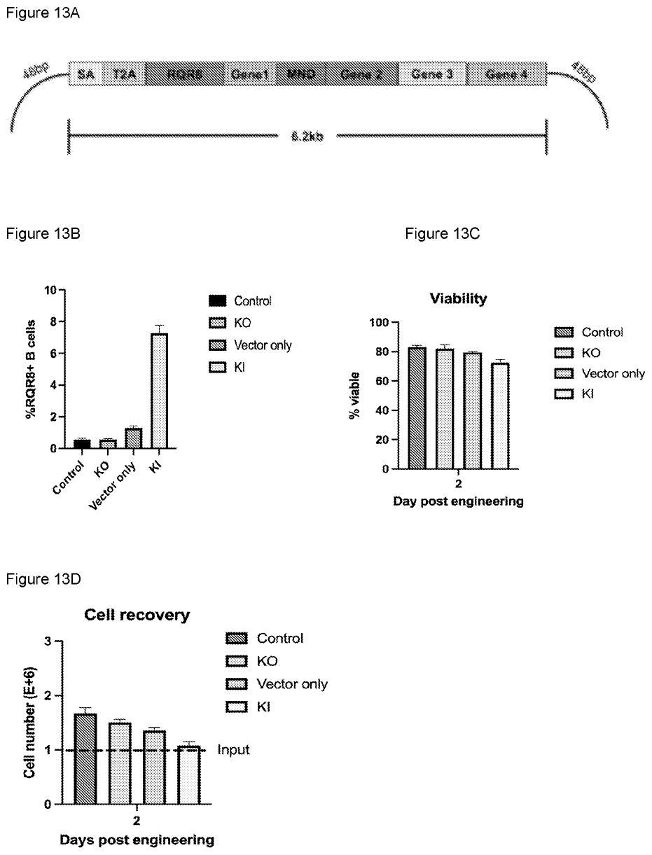

A diagram of the large, 6.2 kb multicistronic construct for targeted integration at AAVS1 locus is shown in FIG. 13A, MesoCAR-2A-RQR8-BGH-MND-CD19-DHFR-GFP-BGH-2, comprising bovine growth hormone, the MND promoter, CD19, DHFR promoter, and GFP. Integration efficiencies when using HMEJ for a targeted knock-in (KI) exceeded >6%. Control B cells were electroporated without nucleic acid, knockout (KO) received Cas9+gRNA and vector only received plasmid alone. (N=2 donors) (FIG. 13B). B cells showed a slight reduction in viability post engineering when compared to electroporated B cells without plasmid (control) (FIG. 13C). However, B cell quantities increased post engineering in all conditions, with control electroporated cells without plasmid having the highest quantity of outgrowth (FIG. 13D).

These results show that large constructs of up to 6.2 kb can be stably integrated into the genome of B cells using the non-viral engineering methods described herein. Additionally, these engineered B cells maintain a high level of viability and continue to proliferate.

It is understood that every embodiment of the disclosure described herein may optionally be combined with any one or more of the other embodiments described herein. Every patent literature and every non-patent literature cited herein are incorporated herein by reference in their entirety.

It is understood, therefore, that this invention is not limited to the particular embodiments disclosed, but is intended to cover all modifications which are within the spirit and scope of the invention as defined by the appended claims; the above description, and/or shown in the attached drawings. Consequently only such limitations as appear in the appended claims should be placed on the disclosure.

### REFERENCES

1. Martino, A T and Markusic, D M (2020). Immune Response Mechanisms against AAV Vectors in Animal Models. Mol. Ther.—Methods Clin. Dev. 17: 198-208.
2. Grimm, D, Lee, J S, Wang, L, Desai, T, Akache, B, Storm, T A, et al. (2008). In vitro and in vivo gene therapy vector evolution via multispecies interbreeding and retargeting of adeno-associated viruses. J. Virol. 82: 5887-911.
3. Laoharawee, K, Johnson, M J, Lahr, W S, Peterson, J J, Webber, B R and Moriarity, B S (2020). Genome Engineering of Primary Human B Cells Using CRISPR/Cas9. J. Vis. Exp.: e61855 doi:10.3791/61855.
4. Yao, X, Wang, X, Hu, X, Liu, Z, Liu, J, Zhou, H, et al. (2017). Homology-mediated end joining-based targeted integration using CRISPR/Cas9. Cell Res. 2017 276 27: 801-814.
5. Briard, B, Place, D E and Kanneganti, T D (2020). DNA sensing in the innate immune response. Physiology 35: 112-124.
6. Hornung, V, Rothenfusser, S, Britsch, S, Krug, A, Jahrsdörfer, B, Giese, T, et al. (2002). Quantitative Expression of Toll-Like Receptor 1-10 mRNA in Cellular Subsets of Human Peripheral Blood Mononuclear Cells and Sensitivity to CpG Oligodeoxynucleotides. J. Immunol. 168: 4531-4537.

What is claimed:

1. A method for genome engineering a B cell or a population of B cells to overexpress a gene of interest comprising introducing into the B cell or B cell population a plasmid, nanoplasmid or mini-circle comprising an expression cassette comprising a homology arm (HA), a polynucleotide encoding a gene of interest, a splice acceptor site, a promoter and a targeting site for a nuclease dependent cleavage system targeting molecule,
wherein the expression cassette is inserted at a locus targeted by the targeting molecule.

2. A method for genome engineering a B cell or a population of B cells to overexpress an endogenous gene comprising introducing into the B cell or B cell population a plasmid, nanoplasmid or mini-circle comprising an expression cassette comprising a homology arm(s) (HA), a splice acceptor site, a promoter and a targeting site for a nuclease dependent cleavage system targeting molecule,
wherein the expression cassette is inserted upstream of a target gene to be overexpressed.

3. The method of claim 1 or 2, wherein the homology arms are between 35 and 1000 nucleotides.

4. The method of any one of claims 1-3, wherein the nuclease dependent cleavage system comprises a CRISPR/Cas system, a Cas-CLOVER system, a zinc-finger nuclease (ZFN) system, a transcription activator like effector nuclease (TALEN) system, or a meganuclease system.

5. The method of claim 4, wherein the CRISPR/Cas system comprises Cas9, Cas12a, Cas13a or Cas13b.

6. The method of any one of claims 1-5, wherein the nuclease dependent cleavage system is a CRISPR/Cas system and the targeting molecule is a guide RNA.

7. The method of claim 6, wherein the method further comprises transfecting the B cell or population of B cells with a Cas protein or polynucleotide encoding a Cas protein and guide RNA molecules that direct integration of the expression cassette to a target locus in the B cell genome.

8. The method of any one of the preceding claims, further comprising introducing into the B cell or B cell population a polynucleotide encoding a biomarker molecule useful to enrich for the B cell or a population of B cells.

9. The method of claim 8, wherein the biomarker molecule comprises a fragment of CD34 and a fragment of CD20.

10. The method of claim 8 or 9, wherein the biomarker polynucleotide is on the same expression cassette as the homology arm(s), splice acceptor site, targeting site for a nuclease dependent cleavage system targeting molecule and promoter of claim 1 or 2.

11. The method of any one of the preceding claims, wherein the gene of interest integrates into the B cell genome via homology directed repair (HDR), homology-mediated end joining (HMEJ) or a combination of HDR/HMEJ.

12. The method of any one of the preceding claims, wherein the introduction of the plasmid or nanoplasmid is by transfection or electroporation.

13. The method of any one of the preceding claims, wherein efficiency of introduction is greater than 15%.

14. The method of any one of the preceding claims, wherein the B cell population has a viability of greater than 60% after 3 days.

15. The method of any one of the preceding claims, wherein the B cell or population of B cells is a primary B cell, a plasma cell, a mature B cell, a B cell line, naïve B cell, memory B cells, or plasma blast.

16. The method of any one of the preceding claims, wherein the plasmid or nanoplasmid comprises the promoter next to or near the gene of interest.

17. The method of claim 16, wherein the promoter is an MND promoter, a CMV promoter, a CAG promoter, a PGK promoter, a EF1A promoter, a FEEK promoter, or a B cell-specific promoter.

18. The method of any one of the preceding claims, wherein the gene of interest is a therapeutic gene or encodes a therapeutic protein.

19. The method of claim 18, wherein the therapeutic gene encodes an enzyme, a cancer antigen, a cytokine, a chemokine, a B cell receptor, a cell surface receptor, or an antibody.

20. The method of any one of the preceding claims, wherein the gene of interest is a donor polynucleotide that corrects a mutated genotype in a subject.

21. The method of any one of the preceding claims, wherein the plasmid or nanoplasmid further comprises a polynucleotide encoding a B cell receptor or fragment thereof.

22. A method of making a gene edited B cell or population of B cells, comprising:
i) contacting a B cell or population of B cells with a plasmid, nanoplasmid or mini-circle comprising an expression cassette comprising a homology arm (HA), a splice acceptor site, a promoter and a targeting site for a nuclease dependent cleavage system targeting molecule, and optionally comprising a polynucleotide encoding a gene of interest;
ii) culturing the B cell or population of B cells of i) in a media that promotes expansion of B cells;
iii) isolating the B cell or population of B cells of ii) based on identification of a marker expressed only on a B cell or population of B cells carrying the plasmid or nanoplasmid;
iv) culturing the isolated cells of iii) in a culture medium to expand the isolated cells expressing the gene of interest.

23. The method of claim 22 further comprising a step of stimulating, proliferating or activating the B cell or population of B cells prior to the contacting step.

24. The method of claim 23, wherein the step of stimulating, proliferating or activating the B cell or population of B cells comprises contacting the cell(s) with one or more of CpG, LPS, CD40L, Anti-IgM, IL-10, IL-15, IL-2, IL-6, IFNα, APRIL or BAFF.

25. The method of any one of claims 22-24, wherein the method produces gene edited B cells with an efficiency of greater than 15%.

26. The method of any one of claims 22-25, wherein the method maintains 60% viability of cells in culture after 3 days.

27. The method of claim 22, wherein the culturing comprises use of culture media supplemented with CpG, or culturing media without CpG.

28. A gene edited B cell or population of B cells made by the method of any one of claims 1-27.

29. A gene edited B cell comprising
i) a heterologous polynucleotide sequence encoding a gene of interest integrated in the B cell genome at a target location mediated by a nuclease dependent cleavage system, wherein the heterologous polynucleotide sequence is also flanked by portions of a homology arm and expressed via an endogenous promoter; and
ii) a heterologous biomarker molecule.

30. The gene edited B cell of claim 29, wherein the B cell is a primary B cell, a plasma cell, a B cell line, naïve B cell, memory B cell, or a plasma blast.

31. The B cell of any one of claim 29 or 30, wherein the gene of interest is a therapeutic gene or encodes a therapeutic protein.

32. The B cell of claim 31, wherein the therapeutic gene encodes an enzyme, a cancer antigen, a cytokine, a chemokine, a B cell receptor, a cell surface receptor, or an antibody.

33. The B cell of any one of claim 29 or 32, wherein the gene of interest is a donor polynucleotide that corrects a mutated genotype in a subject.

34. A method of treating a disease or condition in a subject in need thereof comprising administering to the subject a gene edited B cell or population of B cells of any one of claims 28-33.

35. The method of claim 34, wherein the disease is an enzymopathy, a cancer, a precancerous condition, an infection, or a genetic disorder.

36. The method of claim 34 or 35, wherein the disease is an enzymopathy.

37. The method of claim 35 or 36, wherein the enzymopathy is selected from the group consisting of aspartylglucosaminuria, cholesterol ester storage disease, Wolman disease, metachromatic leukodystrophy, Danon disease, Fabry disease, Farber lipogranulomatosis, Farber disease, fucosidosis, galactosialidosis types I/II, Gaucher disease types I/II/III, globoid cell leukodystrophy, Krabbe disease, glycogen storage disease II, Pompe disease, GM1-gangliosidosis types I/II/III, GM2-gangliosidosis type I, Tay Sachs disease, GM2-gangliosidosis type II, Sandhoff disease, GM2-gangliosidosis, α-mannosidosis types I/II, β-mannosidosis, mucolipidosis type I, sialidosis types I/II, mucolipidosis types II/III, I-cell disease, mucolipidosis type IIIC, pseudo-Hurler polydystrophy, mucopolysaccharidosis type I, mucopolysaccharidosis type II, Hunter syndrome, mucopolysaccharidosis type IIIA, Sanfilippo syndrome, mucopolysaccharidosis type IIIB, mucopolysaccharidosis type IIIC, mucopolysaccharidosis type IIID, mucopolysaccharidosis type IVA, mucopolysaccharidosis type IVB Morquio syndrome, mucopolysaccharidosis type VI, mucopolysaccharidosis type VII, Sly syndrome, mucopolysaccharidosis type IX, multiple sulfatase deficiency, neuronal ceroid lipofuscinosis, CLN1 Batten disease, CLN2 Batten disease, Niemann-Pick disease types A/B, Niemann-Pick disease, Niemann-Pick disease type C1, Niemann-Pick disease type C2, pycnodysostosis, Schindler disease types I/II, and sialic acid storage disease, hemophilia A, hemophilia B, Christmas disease, and Factor VII deficiency.

38. The method of claim 37, wherein the enzymopathy is mucopolysaccharidosis type I (MPS I) and the gene of interest is iduronidase.

39. The method of claim 35, wherein the cancer is a solid tumor or a blood cancer.

40. The method of claim 35 or 39, wherein the cancer is selected from the group consisting of acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head and neck cancer, Hodgkin's lymphoma, Kaposi's sarcoma, kidney cancer, liver cancer, lung cancer (small and non-small cell), malignant peritoneal effusion, malignant pleural effusion, melanoma, mesothelioma, multiple myeloma, neuroblastoma, glioma, non-Hodgkin's lymphoma, osteosarcoma, ovarian cancer, ovarian (germ cell) cancer, pancreatic cancer, penile cancer, prostate cancer, retinoblastoma, skin cancer, soft tissue sarcoma, squamous cell carcinomas, stomach cancer, testicular cancer, thyroid cancer, trophoblastic neoplasms, uterine cancer, vaginal cancer, cancer of the vulva, and Wilms's tumor.

41. The method of claim 35, wherein the genetic disorder is selected from the group consisting of muscular dystrophy, cystic fibrosis, Sickle cell anemia, β-thalassemia, a lysosomal storage disorder, Adenosine Deaminase Deficiency, Severe Combined Immunodeficiency (SCID), Retinitis Pigmentosa, macular degeneration, and Wiskott-Aldrich Syndrome.

42. The method of any one of claims 35-41, wherein the B cell is a primary B cell, a plasma cell, a mature B cell, a naïve B cell, a memory B cell, or a plasma blast.

43. The method of any one of claims 35-42, wherein the B cell is first isolated from the subject to be treated and then genetically modified according to the method of any one of claims 1-27.
